(19) United States
(12) Patent Application Publication
(10) Pub. No.: US 2009/0123583 A1
(43) Pub. Date: May 14, 2009

(54) Method for Obtaining Fruit Wax

(76) Inventors: Johann Wiesmuller, Garching (DE); Stephan Pilz, Fellbach (DE)

Correspondence Address: FULBRIGHT & JAWORSKI, LLP, 666 FIFTH AVE, NEW YORK, NY, 10103-3198, US

(21) Appl. No.: 11/989,351

(22) PCT Filed: Aug. 4, 2006
(86) PCT No.: PCT/EP2006/007756

§ 371 Date: Jan. 24, 2008

(30) Foreign Application Priority Data
Aug. 6, 2005 (DE) 10 2005 037 210.4

(51) Int. Cl. A61K 36/00; C11B 1/10

(52) U.S. Cl. 424/777; 554/20

(57) ABSTRACT

The invention relates to a method for obtaining fruit wax from plant constituents, said method being carried out essentially using compressed C₂-C₄ hydrocarbons. Unlike previous extraction methods with supercritical CO₂, the inventive method can be carried out at low pressures and with a reduced extraction means throughput. Residues from the fruit treatment, especially peel from the juicing process, are used as starting materials. Preferred extraction agents are ethane, propane, butane and the mixtures thereof, the extraction itself being carried out in batches at pressures of <50 mPa and temperatures of ≦70° C., with an extraction agent throughput of between 4 and 20 kg/kg of starting materials. The extracted fruit wax is outstandingly suitable for cosmetic or pharmaceutical preparations, especially for the care and treatment of the skin.

The present invention relates to a method for obtaining fruit waxes from plant constituents by extraction with the help of compressed gases.

Fruit waxes, in particular on a predominantly natural basis, are increasingly important starting materials for obtaining active ingredients which are used in particular as protective and care active ingredients by the cosmetics industry. The advantage of such components is in particular to be regarded as being the fact that they are renewable raw materials which, from ecological and economic points of view, makes them appear extremely valuable. In addition, the use of natural ingredients significantly increases consumer acceptance since consumers have in the meantime high ecological awareness and select products for body care and healthcare extremely critically.

Various terms are used synonymously specifically in the area of waxes and paraffins, where, according to the generally accepted definition, “waxes” are associated with lipophilic substances of plastic consistency, i.e. wax-like nature. Waxes are fatty acid esters of long-chain or cyclic monohydric or dihydric alcohols. These waxes are mostly of vegetable or animal origin, as a result of which they are typical natural products. Strict delimitation of the vegetable and animal waxes on the basis of the fatty acids and fatty alcohols involved in their cultivation cannot be undertaken. However, it is indisputable that montanic acid, palmitic acid and stearic acid are typical fatty acids contributing to these natural waxes. On the side of the alcohols, cetyl alcohol and ceryl alcohol in particular are to be mentioned here.

True natural waxes of vegetable origin are, for example, palm leaf waxes, such as carnauba wax, palm wax, raffia wax, ouricoury wax, grass waxes, such as, for example, candelilla wax, esparto wax, fiber wax and sugarcane wax; berry and fruit waxes are, for example, Japan wax, bayberry wax and myrtle wax. The best known example of a true natural wax of animal origin is beeswax, which consists primarily of miricyl palmitate, i.e. of a palmitic acid esterified with miricyl alcohol. Also known are Chinese insect waxes, shellac wax and wool wax, as can be obtained, for example, from sheep wool.

Fruit waxes and in particular apple waxes are already used by the cosmetics industry in diverse areas of application and in particular in skincare and hair care products and, in shampoos, rinses and hair treatments, protect the skin, but also the hair as a thin film against dryness and leeching out. By way of example, mention may be made here of WO 03/053 394 A2.

The raw material source for these apple waxes is primarily pomaces, i.e. dried residues from the manufacture of apple juice and pectin. The apple waxes present in this pomace originate from the fruit peels of juice apples. After these fruit waxes have been isolated from the pomace and purified of chlorophyll and pesticide residues, an odor-neutral raw material is generally available. This raw material is accessible without further-reaching transformation, i.e. it can be purified and isolated by purely physical methods such as, for example, adsorption, filtration and distillation. The residue from the fruit wax isolation can, like the fruit-wax-containing pomace as well, be used as cattle feed.

The already discussed isolation of the fruit waxes has hitherto taken place with the help of supercritical out with the help of compressed C₂-C₄-hydrocarbons.

In practice, it has been found that with this novel method according to the invention, not only could the objective be achieved but that fruit waxes can surprisingly be obtained with it in sometimes significantly increased yields. These yields exceed the yields as are obtainable with the help of supercritical carbon dioxide according to the prior art.

Furthermore, the fruit waxes obtainable with the method according to the invention are of a quality which renders them accessible to new fields of application.

As regards the starting material, it has proven favorable to use residues from the processing of fruit for the method according to the invention, where in particular fruit peels, e.g. from the production of juice and here, particularly preferably, pomaces and primarily wet pomaces are suitable. The fruit pips and any pieces of rind, leaves, stems and woody tissue are particularly preferably separated off before the extraction.

Pomaces, such as, for example, apple pomaces, generally consist of 10 to 15% by weight of pectin, of 20 to 30% by weight of sugars (fructose, glucose, sucrose) and of 55 to 70% by weight of non-pectin substances, such as, for example, peels, pips etc. For the actual extraction, it is recommended to dry the wet pomaces, as a result of which the actual extraction method can be carried out significantly more economically. Preferably, the water content of the extraction material is ≦15% by weight, particularly preferably 1 to 10% by weight.

As extraction agents, the present invention considers ethane, propane and butane, and mixtures thereof. In this connection, if necessary, entrainers such as carbon dioxide, which is a solvent which has been able to be used quite safely for decades for the treatment of food. The corresponding extraction and/or purification with compressed carbon dioxide gives apple waxes in yields of about 2% in cosmetics-grade quality.

As already indicated natural waxes are not a single substance class, but a mixture of different substance classes. These natural extracts comprise, as main components, wax esters, hydrocarbons, fatty acids and other compounds, such as, for example, triglycerides, xanthophylls and terpenes.

On account of the heterogeneous composition of the natural extracts used hitherto for isolating fruit wax and the known risk-free properties of carbon dioxide, the use of supercritical CO₂ has hitherto been the extraction agent of choice. In this connection, however, the low selectivity has been accepted as a trade-off. Also, the CO₂ extraction requires high pressures and a high CO₂ throughput, which makes such processes expensive. Since the cosmetics industry is increasingly demanding, irrespective of the particular field of use, purer products with a defined component spectrum, extraction using supercritical carbon dioxide no longer suffices, particularly also from economical points of view.

On account of this disadvantage of the prior art, the object set for the present invention is to provide a novel method for obtaining fruit waxes from plant constituents by extraction with the help of compressed gases which meets the further increased requirements by the processing industry and by the consumer. The novel method should be commercially implementable in the simplest possible manner and provide fruit waxes in improved quality.

This object was achieved with a method which is carried dimethyl ether and alcohols can also be added to the compressed hydrocarbon (mixture) used in each case as extraction agent, in which case these are then preferably used in amounts of from 0.5 to 50% by weight.

As regards the extraction parameters, the present invention recommends pressures of <50 MPa and temperatures of ≦70° C., with a pressure range between 0.5 and 10 MPa and temperatures between 20 and 35° C. being regarded as particularly preferred.

Depending on the particular starting material (for example its moisture content), the method according to the present invention should be carried out with an extraction agent throughput of from 4 to 20 kg/kg of starting material, with a throughput of between 5 and 10 kg/kg of starting material being regarded as preferred.

To separate off the extract, a pressure reduction to 5 to 10 bar at temperatures between 40 and 48° C. is usually carried out.

Although the proposed method can also be carried out continuously, for practical reasons, the invention envisages carrying out the method batchwise since, in so doing, the process parameters can be matched to the particular starting material, its nature and composition in a readily reproducible manner.

Besides the actual method itself, the present invention also claims the use of a fruit wax extracted using the proposed method in cosmetic or pharmaceutical preparations and in particular for the care and treatment of the skin and appendages thereof, which is to be understood primarily as meaning hair and also toenails and fingernails. Within the scope of the present invention, the term “skin” is naturally to be understood firstly as meaning the skin itself, but also mucous membranes and the skin appendages, where these comprise living cells. Here, in particular hair follicle, hair root, hair bulb and the ventral epithelium of the nail bed, sebaceous glands and sweat glands are to be understood. The use according to the invention includes the care, and therapeutic and nontherapeutic treatment.

With the novel method for the extraction of fruit waxes with the help of C₂-C₄-hydrocarbons, it is possible to obtain natural fruit waxes in improved quality and increased yields under extremely economical conditions from starting materials which are usually waste materials and are now used as secondary raw materials.

The example below demonstrates the advantages of the claimed method.

### EXAMPLE

In the production of apple juice from juice apples, apple pomace is produced which also contains the residues of apple-wax-containing fruit peels.

After drying the apple pomace to moisture contents of <15% by weight, the constituents such as pips, stems and leaves were removed. The remaining residue was extracted with liquid propane in an autoclave at 30 bar and 35° C., the extraction agent throughput being on average 7.3 kg/kg of starting material. The extract obtained in this way was separated off after the pressure had been reduced at 8 bar and 46° C.

In this way, a yellow, odor-neutral apple wax extract was obtained in a yield of about 2.5%.

1-9. (canceled)

10. A method for extracting a fruit wax from a plant constituent comprising extracting said wax by contacting said plant constituent with at least one of compressed propane and compressed butane, said extracting being carried out at a pressure of <50 MPa and a temperature of ≦70° C.

11. The method of claim 10, wherein said plant constituent is a fruit peel or a pomace.

12. The method of claim 11, wherein said pomace is a wet pomace.

13. The method of claim 10, further comprising adjusting said pressure to from 0.5 MPa to 10 MPa and said temperature to from 20° C. to 35° C.

14. The method of claim 10, further comprising adding an entrainer to said compressed propane or compressed butane.

15. The method of claim 14, wherein said entrainer is an alcohol or dimethyl ether.

16. The method of claim 14, comprising adding said entrainer to said compressed propane or compressed butane in an amount of from 0.5 to 50% by weight of said compressed propane or compressed butane.

17. The method of claim 10, comprising extracting said fruit wax in a process whereby a throughput of from 4 kg/kg to 20 kg/kg of starting material are used.

18. The method of claim 17, wherein said throughput is from 5 kg/kg to 10 kg/kg.

19. The method of claim 10, comprising a batchwise process.

20. The method of claim 10, wherein said plant constituent comprises residue from fruit processing.

21. Composition comprising the fruit wax produced via the method of claim 10, and a cosmetic or pharmaceutically acceptable carrier.
